(19) United States
(12) Patent Application Publication
(10) Pub. No.: US 2010/0313898 A1
(43) Pub. Date: Dec. 16, 2010

(54) APPARATUS AND METHODS FOR TREATING SLEEP RELATED DISORDERS

(76) Inventors: Ronald F. Richard, Escondido, CA (US); Paul L. Edwards, Encinitas, CA (US); Patrick Dunne, Fullerton, CA (US); Adrian Knight, Shawnee, KS (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/780,812

(22) Filed: May 14, 2010

Related U.S. Application Data
(60) Provisional application No. 61/178,664, filed on May 15, 2009.

(51) Int. Cl. A61F 5/56; A62B 7/00

(52) U.S. Cl. 128/848; 128/200.24

(57) ABSTRACT

This disclosure relates generally to a method and an apparatus for delivering a gas to a subject in need thereof, for instance, for the treatment of an unhealthy condition, such as sleep apnea. The method may include sensing the onset of a subject's inhalation and delivering a gas, such as a pulse of gas, in response to the subject's inhalation. For instance, the method may include providing the subject with a gas delivery device configured for delivering a quantity of gas to the subject, such as in response to the subject's need, and instructing the subject to use the device. The device may include a mechanism for generating and delivering the quantity of gas to the subject, as well as a sensor for sensing the subject's need for the gas. A controller for controlling the delivery of the gas to the subject in response to the subject's sensed need for the gas may also be included. The device may further include a cannula to facilitate delivery of the gas to the subject.

### CROSS REFERENCE TO RELATED APPLICATIONS

This application claims the benefit of priority under 35 U.S.C. §119 to U.S. Provisional Patent Application Ser. No. 61/178,664, filed May 15, 2009, entitled, “System and Method for Treating Mild-to-Moderate Obstructive Sleep Apnea Using Pulsed Air/Concentrated Oxygen,” the entire disclosure of which is incorporated by reference herein.

### FIELD

This disclosure relates generally to a method and an apparatus for treating sleep related disorders, such as sleep apnea.

### BACKGROUND

Sleep apnea is a sleep disorder characterized by a series of pauses in breathing during sleep. Each individual episode is called an apnea. The apnea may last for a variable period of time such that one or more breaths are missed. These episodes occur repeatedly during sleep. The typical apneic event includes about a 10 second interval between breaths, which becomes clinically significant where five or more episodes occur per hour.

There are three distinct forms of sleep apnea: central, obstructive, and a combination of central and obstructive. Central sleep apnea (CSA) is characterized by breathing that is interrupted by a lack of respiratory effort. Obstructive sleep apnea (OSA), involves apnea caused by an obstruction of the airway. Specifically, breathing is interrupted by a physical block to airflow regardless of respiratory effort. In mixed sleep apnea, there is a transition from central to obstructive features during the apneic events themselves.

Many people suffer from sleep apnea. Typically sleep apnea can cause a person to stop breathing over a hundred times during a nights sleep. These interruptions in oxygen supply may result in de-saturation, hypoxia, and generally result in an overall poor quality of sleep. A person suffering from symptoms of sleep apnea may undergo a sleep study to determine if the apnea poses a clinical problem. The sleep study is non invasive and may be performed at an outpatient facility. This study enables a physician to examine the person during sleep so as to determine the severity of the sleep disorder.

As known in the art, the typical treatment regime for sleep apnea involves the application of continuous positive airway pressure (“CPAP”). The CPAP device includes a compressor and/or blower that is connected to a nasal mask via a tube attached thereto. CPAP acts like a continuous pneumatic splint. It functions by maintaining the patency of the upper airway thereby allowing the patient to maintain somewhat of a regular breathing pattern.

There are several complications with the use of the CPAP system for the treatment of sleep apnea. For instance, the nasal mask used in the CPAP system for the treatment of sleep apnea is bulky and uncomfortable. Specifically, the CPAP system requires the maintenance of a positive pressure throughout the delivery system, and as such the nasal mask must cover both the nose and mouth in order for the system to properly function. The nasal masks employed by the CPAP system are big, stiff, and rigid, which makes sleeping while wearing such a mask difficult and, thus, adherence is low. Additionally, the CPAP system is configured for the continuous delivery of gas during both the inhalation and exhalation stages of the patient's sleep cycle. As gas delivered during a large portion of the inhalation phase as well as all of the gas delivered during the exhalation phase is wasted. The CPAP system, therefore, is inefficient. In view of these complications, many patients discontinue use of the CPAP system.

Accordingly, the present disclosure is directed to an apparatus and method for treating sleep related disorders, such as sleep apnea, in a manner that both improves patient adherence as well as increases efficiency.

### SUMMARY

In one aspect, a method for delivering a gas to a subject in need thereof while sleeping is provided. The method may include sensing the onset of a subject's inhalation and delivering a gas, such as a pulse of gas, in response to the subject's inhalation. In certain instances, the waveform of the pulse of gas to be delivered may be shaped to meet the specific needs of the subject. For instance, the sharpness, amplitude, and/or length of the waveform to be delivered may be shaped so as to ensure the patency of the upper airwaves of the subject while sleeping.

Accordingly, the gas to be delivered may be administered in one or more pulses. The sharpness, amplitude (e.g., size) and/or the length (e.g., duration) of the pulse may be adjusted, for instance, so as to control the shape of the gas flow wave to meet the needs of the subject. In certain instances, a plurality of pulses may be delivered in a cyclical manner, such as in accordance with a subject's determined breathing cycle.

The method may further include sensing a change in or cessation of breathing, e.g., an apneic event, and in response thereto automatically adjusting the waveform being delivered so as to prevent further untoward changes in breathing and thereby promoting a more restful sleep. In certain embodiments, if further changes in breathing and/or apneic events occur, a continuous flow of gas may be delivered, for example, until a more normalized breathing pattern is established, in which instance a pulsed delivery of gas may be resumed. Accordingly, the method may include providing a subject with a gas delivery device configured for delivering a quantity of gas to the subject, such as in response to the subject's need while sleeping, and instructing the subject to use the device.

In one aspect, a method for treating an unhealthy sleep condition is provided. The method may include examining the subject's sleeping pattern, for instance, so as to determine the cycle as to when the subject inhales and when the subject exhales, and/or the subject's tidal volume while sleeping, as well as determining the occurrence of one or more sleep apneic events. The examination may include delivering a quantity of gas to the subject while sleeping and determining the characteristics of the waveform of the gas being delivered that appear to be beneficial to the subject. By “beneficial to the subject” is meant that those characteristics of the waveform that reduce the number of apneic events experienced by the subject while sleeping are determined and/or are theoretically optimized. The method may then further include providing the subject with a device configured for delivering a quantity of gas to the subject in accordance with one or more of the above identified predetermined characteristics. The device may further be configured for delivering the gas continuously, in response to the subject's need, or for switching between the two as needed.

In certain embodiments, the gas is to be delivered at the onset of a determined inhalation event. The determined inhalation event may be sensed or may be predetermined, for instance, as a result of a sleep study. For example, in certain instances, the onset of the inhalation event may be detected as a drop in pressure, wherein the gas may be delivered in response to the sensed drop in pressure. In other instances, the gas may be delivered in accordance with the subject's determined sleeping pattern, for instance, at the onset of an expected inhalation phase that has been predetermined by the study. In certain embodiments, the device may be configured for determining a running inhalation pattern in situ and delivering the gas in response to the running inhalation pattern. The method may additionally include instructing the subject to use the device while sleeping and/or providing the subject with instructions as to how to properly use the device.

The gas may be delivered for any suitable purpose, such as for treating a subject suffering from a symptom associated with one or more of sleep apnea, snoring, asthma, allergies, inflammation, hypertension, cardiovascular complications, stroke, type II diabetes, fatigue, sleepiness, and the like. In certain instances, the gas may be delivered in conjunction with a medicament, such as a medicament in aerosol form. In other instances, the gas may be delivered for the purpose of delivering a positive pressure to the oral cavity, such as the upper airway, of the subject. For instance, in some instances, the gas to be delivered may be configured so as to act like a pneumatic splint enhancing the patency of the upper airway. In further instances, the gas may be delivered for the purpose of stimulating the hypoglossal nerve. For instance, in some instances, the gas to be delivered may be configured to activate one or more of the baroreceptors of the upper airways in a subject. In some instances, the gas may be delivered for the purpose of synchronizing the breathing of the subject.

In one aspect, a device for delivering a quantity of gas to a subject is provided. The device may be configured such that the quantity of gas to be delivered is in response to the subject's need. The device may include a mechanism for generating and delivering the quantity of gas to the subject, as well as a sensor for sensing the subject's need for the gas. A controller for controlling the delivery of the gas to the subject is also included. The controller may be configured for delivering the gas in response to the sensed need for the gas, in accordance with a predetermined pattern, a running determination pattern, continuously, or a combination thereof. The device may further include a specialized cannula, such as a high flow nasal cannula, to facilitate delivery of the gas to the subject. In certain instances, the device may be configured for delivering a quantity of gas to a subject in pulses or continuously, and in certain instances, may be configured such that one or more characteristics of a pulse to be delivered is capable of being modulated, for example, to shape the pulse to meet the specific needs of the user.

It is to be noted that the subject matter disclosed herein provides systems, apparatus, and methods which may include a computer program product and/or system, for treating a sleep related disorder. Accordingly, articles are also described that comprise a tangibly embodied machine-readable medium embodying instructions that, when performed, cause one or more machines (e.g., computers, gas delivery devices, etc.) to result in operations described herein. Similarly, computer systems are also described that may include a processor and a memory coupled to the processor. The memory may include one or more programs that cause the processor to perform one or more of the operations described herein. Other features and advantages of the subject matter described herein will be apparent from the description and drawings, and from the claims set forth herein below.

### BRIEF DESCRIPTION OF THE DRAWING

These and other aspects will now be described in detail with reference to the following drawings.

FIGS. 1A and 1B are block diagrams of different embodiments of a titration system for treating mild-to-moderate sleep apnea using a pulse-mode air/concentrated oxygen gas system.

FIGS. 2A and 2B are an illustration of an exemplary bellows for use in a device of the disclosure.

FIGS. 3A, 3B, and 3C are an illustration of an exemplary piston for use in a device of the disclosure.

FIG. 4 is a block diagram of an embodiment of a pulse-mode air/concentrated oxygen gas system of the titration system illustrated in FIG. 1.

FIG. 5 is a flow chart of an exemplary titration method in accordance with an embodiment of the invention.

FIG. 6 is a flow chart of an exemplary method for treating mild-to-moderate sleep apnea using a pulse-mode air/concentrated oxygen gas system.

FIG. 7 is a block diagram illustrating an example computer system that may be used in connection with various embodiments described herein.

Like reference symbols in the various drawings indicate like elements.

### DETAILED DESCRIPTION

The subject matter described herein relates generally to gas concentrators and/or systems, such as pulse or continuous oxygen concentrator systems, titration systems, titration methods, and methods for treating unhealthy conditions. For instance, a method and an apparatus for delivering a gas, such as air or oxygen, to a subject in need thereof, for instance, intermittently, for the treatment of an unhealthy condition, such as sleep apnea. The method may include sensing the onset of a subject's inhalation and delivering a gas, such as a pulse of air or oxygen, in response to the subject's inhalation. In this manner, a pulse of air or oxygen may be delivered periodically to the subject, for example, during the inhalation phase of the subject's breathing cycle.

There are several advantages achieved by such periodic and/or pulsitile delivery. For instance, where the gas to be delivered is air or oxygen, a subject in need thereof can primarily only make use of the gas during the first part of the inhalation phase of breathing. Gas delivered beyond this phase, therefore, is largely wasted. Accordingly, the present methods and devices which can deliver a gas to a user periodically, such as in response to a subject's need, represent a significant advancement over those devices known in the art that simply deliver a gas continuously to a user because a large portion of that gas is wasted. Such devices, therefore, are not as efficient as the presently described devices and consequently the present devices advantageously conserve energy over the devices known in the art.

Additionally, as described herein below, the methods and apparatus of the present disclosure also advantageously function, in part, to contour the shape of the waveform of the flow of the gas being delivered. For instance, as described herein the methods and devices of the present disclosure may be configured to modulate one or more of the rise time, size, duration, and therefore the shape of the waveform of the flow of the gas being delivered. For example, in accordance with the methods and devices described herein the shape of the waveform of a bolus or stream of gas to be delivered to a subject may be shaped, for instance, so as to meet the particular needs of the subject. This may be accomplished by configuring the controller to control one or more of the motor and gas generator and/or the valve so as to shape the waveform.

Further, an apparatus of the disclosure may be configured for, and thus, the methods may include, sensing a change in or cessation of breathing, e.g., an apneic event, and in response thereto automatically adjusting the waveform being delivered so as to prevent further untoward changes in breathing and thereby promoting a more restful sleep. In certain embodiments, if further changes in breathing and/or apneic events occur, a continuous flow of gas may be delivered, for example, until a more normalized breathing pattern is established, in which instance a pulsed delivery of gas may be resumed.

In such manners, the methods and devices presented herein may be used, for example, to prevent and/or treat symptoms associated with one or more of obstructive sleep, sleep apnea, snoring, asthma, allergies, inflammation, hypertension, cardiovascular complications, stroke, type II diabetes, fatigue, and sleepiness. For instance, if sleep apnea persists, a person may manifest symptoms associated with one or more of the conditions set forth above.

Accordingly, it has been determined that the architecture of a subject's upper airway changes during sleep. For instance, the muscles underlying the airways may relax, causing the tissue of the airway to constrict, thereby closing off the airway, and thus causing an apneic event. In one aspect, therefore, the devices and methods disclosed herein have been developed so as to produce a more patent airway architecture during sleep. For instance, without being held to theory, it is believed that by providing a quantity of gas to the airway, which quantity of gas has a patterned waveform, one or more of the hypoglossal nerve and the baroreceptors proximal to the airway may be stimulated, and/or a positive pressure may be delivered to the airway so as to act as a pneumatic splint, thereby enhancing the patency of the airway. This is an advance over other devices known in the art wherein a gas is delivered continuously under a constant pressure.

Hence, in one aspect, the disclosure is directed to delivering a flow of a gas to a subject, wherein the wave of the flow is modulated, for instance, in accordance with both the need of the subject and/or one or more predetermined parameters so as to prevent or treat an unhealthy condition. The shape of the flow of the gas wave can be modulated by modulating the rise or ramp up time of the delivery, the amplitude or volume and/or the length or duration of the delivery. For example the shorter the rise time, the sharper the initial delivery will be, and the slower the rise time the more blunted will be the delivery. Further, the more gas delivered the higher the amplitude of the wave and consequently the higher the pressure will be, and a delivery of a lesser amount of gas will result in a lesser amplitude and a lesser amount of pressure. Further still, the longer the time period during which the gas is delivered, the longer the duration will be, and vice-versa, the shorter the time period during which the gas is delivered, the shorter the duration will be. Thus, where in conventional devices the pulse is typically a square wave, the waveform of the gas to be delivered in accordance with the methods and devices described herein may be contoured, e.g., the leading and falling edges may be rounded, for instance, in response to the subject's need. Hence, the waveform may vary, e.g., from a square wave to a more sinusoidal waveform, in accordance with a subject's determined need.

Additionally, the gas may be delivered in periodic pulses, wherein for any individual pulse, once a maximum amplitude has been reached, the waveform may be oscillated during at least a portion of the duration so as to give the waveform a jagged configuration at the upper end. Without being held to theory, it is believed that this jagged waveform configuration may enhance activation of the baroreceptors and thereby enhance the patency of the underlying architecture of the upper airways. In certain instances, such activation of the baroreceptors may result in ventilatory Long Term Facilitation (LTF), thereby reducing obstructive sleep apnea events in a subject.

The method includes providing the subject with a gas delivery device, as provided herein, and instructing the subject to use the device. The device may include a mechanism for generating and delivering the quantity of gas to the subject, as well as a sensor for sensing the subject's need for the gas. A controller for controlling the delivery of the gas to the subject in response to the subject's sensed and/or determined need for the gas may also be included.

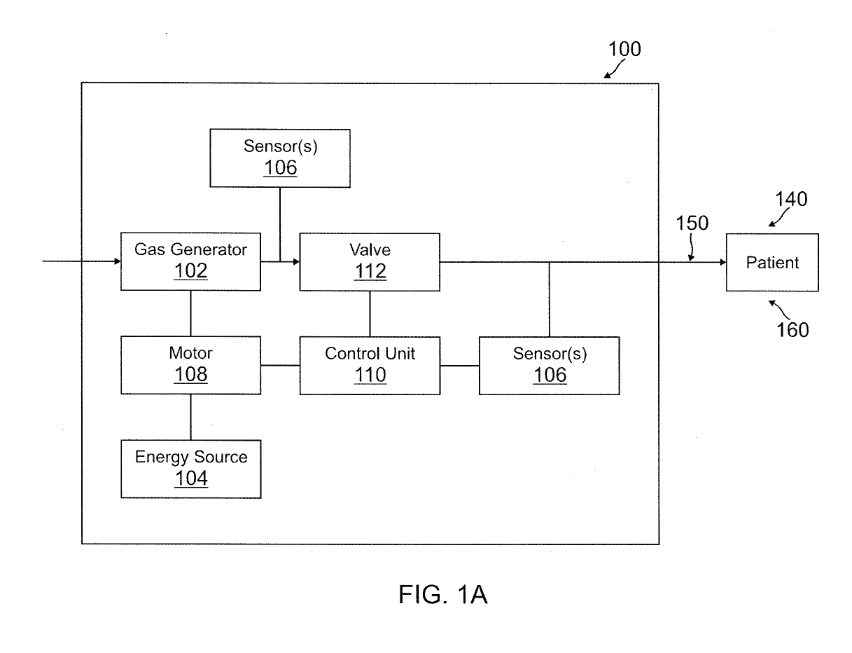

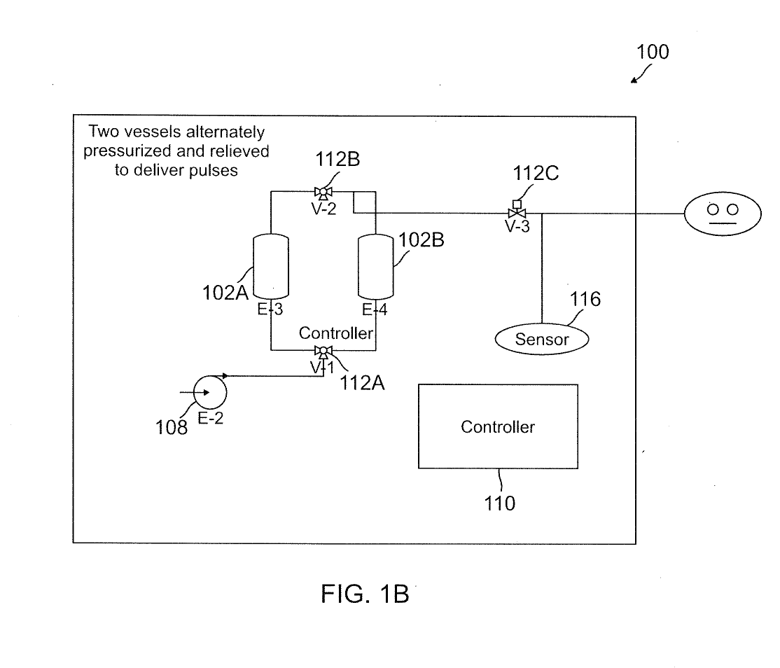

The details of one or more variations of the subject matter described herein are set forth in the accompanying drawings and the following description. For instance, as set forth above, a method for providing a gas to a subject is provided. The method includes providing the subject with a suitable gas delivery device, such as that illustrated in FIG. 1, and instructing the subject to use the device. A suitable device of the system may be designed such that the weight of the device may be in the range from about 4 to about 20 lbs, such as from about 6 to about 18 lbs, for instance, 8 lbs to about 12 lbs, including about 10 lbs or less.

Accordingly, FIG. 1A provides a gas delivery system 100 for use in accordance with the methods described herein. The gas delivery system 100 includes a gas generator 102, e.g., a bellows, a piston, or the like, which is operatively connected to a motor 108, which motor is powered by a suitable energy source 104, and a sensor 106, such as an output sensor configured for sensing one or more conditions of the subject and/or environment. A suitable energy source 104 may be an AC/DC power source, rechargeable battery, battery pack, fuel cell, and the like. One or more valves 112 may also be included. A suitable sensor may be a flow sensor, an ultrasonic flow sensor, a gas temperature sensor, atmospheric pressure sensor, a humidity sensor, a breath sensor, breath detection sensor, pressure sensor, and the like. Additionally, the device may also include a controller 110 for controlling the flow of the gas to the user. In certain instances, the gas delivery system 100 is configured for delivering a gas to a user either continuously or in one or more pulses. For instance, a bolus of gas may be delivered to a user in a series of pulses.

Hence, in certain instances, the system is configured for delivering a pulse of a gas, such as air or oxygen, intermittently, for instance, in response to a users breathing or in accordance with a predetermined pattern thereof. In certain instances, therefore, the device may be configured for delivering gas to a user in response to a sensed need therefore, e.g., periodically, or in a cyclical manner in accordance with a predetermined pattern, or continuously and/or may be configured for switching between intermittent mode, predetermined delivery mode, and/or continuous mode, such as in response to a predetermined event. For instance, in certain instances, if a subject experiences an apnea event, the system and/or method may be configured so as to default into a correction mode and/or a predetermined continuous flow rate, as described below. Once the subject resumes spontaneous breathing, the subject's breath rate will again trigger the system thereby driving the flow of the gas to the subject.

Any suitable gas generator 102 may be employed so long as it is capable of generating at least a pulse of gas to be delivered to a user. For instance, in certain embodiments, the gas generator is capable of generating a bolus of gas, the flow and/or pressure of which bolus may be shaped so as to meet the particular requirements of the user prior to delivery thereto. A suitable gas generator may include one or more of a bellows, a piston, an adjustable speed blower, a blower and valve combination, a pump up tank and solenoid or proportional valve system, a compressed oxygen or air cylinder, e.g., with a conserving device, and the like. Other suitable mechanisms for providing gas to a user may also be provided, for example, suitably configured volume with a moving element, e.g., stereo speakers, attached to a motive element, or a compressed gas tank in combination with an electronic or pneumatic conserver and/or adjustable valve assembly. Any suitable motor may be used to drive the gas generator, such as an electric motor, for instance, a synchronous motor, stepper motor or a continuous motor, a linear motor, a rotary motor, or the like.

The controller 110 may be any suitable mechanism for controlling the flow of the gas to the user. In certain instances, the controller 110 is configured for interacting with a sensor, such as a pressure sensor, 106 so as to control the motor 108 powering the gas generator 102 and thereby controlling the flow of the gas to the user. The flow of the gas to the user may be continuous, intermittent, or a combination of the two over a given period of time.

For instance, where the flow is intermittent, such as when a bolus of gas is to be delivered in pulses, the rise time, size, e.g., volume, height, shape, and timing of the pulse may all be controlled so as, for example, to modulate the shape of the wave flow to match the particular needs of the user. The speed of ramp up, the dose volume, the length of delivery, as well as the number of pulses may, therefore, be modulated by the suitable programming of the controller. Accordingly, the flow of gas to the user may be breath, e.g., inhalation, activated and/or synchronized to the breathing of the user and/or in accordance with one or more predetermined patterns. A suitable controller may be, for instance, a microprocessor.

Additionally, a sensor may be included. A suitable sensor may be used so long as the sensor is capable of being associated with a controller so as to communicate therewith. For instance, the sensor may be a pressure sensor, which sensor may be configured for detecting a pressure differential, e.g., a drop in pressure, such as a pressure drop that is indicative of the onset of an inhalation event, or for detecting an output flow in the device and communicating the same with the controller 110. For example, in certain instances, the pressure sensor determines that a subject is beginning a breath and activates the controller which controls the opening and closing of a flow delivery valve. A suitable pressure sensor may be one that is capable of detecting a drop in pressure that is a negative pressure, such as in the range of about 0.005 or less to about 0.95 cmH₂O or more, for instance, as from about 0.05 to about 0.90 cmH₂O, including from about 0.10 or 0.20 cmH₂O to about 0.85 or 0.75 cmH₂O. In one embodiment, a flow meter may be provided so as to measure air flow and communicate the same to the controller, which controller can control the gas generator and/or one or more valves of the system so as to control the flow of gas to be delivered in response to the flow meter and/or pressure sensor. The flow meter may be used in a feedback loop so as to control the flow delivery valve thus enabling the shaping of the pulse, as described herein. Other suitable sensors may be included such as various environmental sensors, for instance, a temperature, other pressure, and/or a humidity sensor can be used to compensate the pulses for varying environmental conditions.

FIG. 1B provides another embodiment of a gas delivery system 100. The system includes a motor 108, which motor is powered by a suitable energy source 104, and a sensor 106. A plurality of valves 112A, 112B, and 112C are also be included. A controller 110 for controlling the flow of the gas to the user is also provided. In certain instances, the gas delivery system 100 includes a one or more, e.g., a plurality of tanks 102A and 102B. The tanks are pressurized by a gas generator 102, such as a pump and/or a compressor. The pressure can be relieved through one or more delivery valves 112B and/or 112C thus providing a bolus of gas to the subject, which gas can be shaped by the delivery valves. As depicted the two tanks 102A and 102B can alternately be pressurized and de-pressurized to provide a series of pulses. In certain instances, while one tank is being filled the other may be relieved

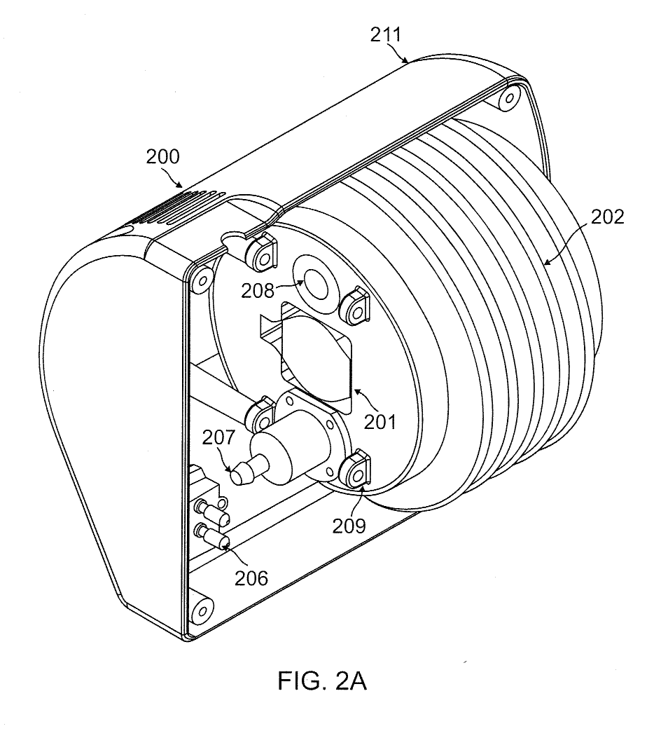

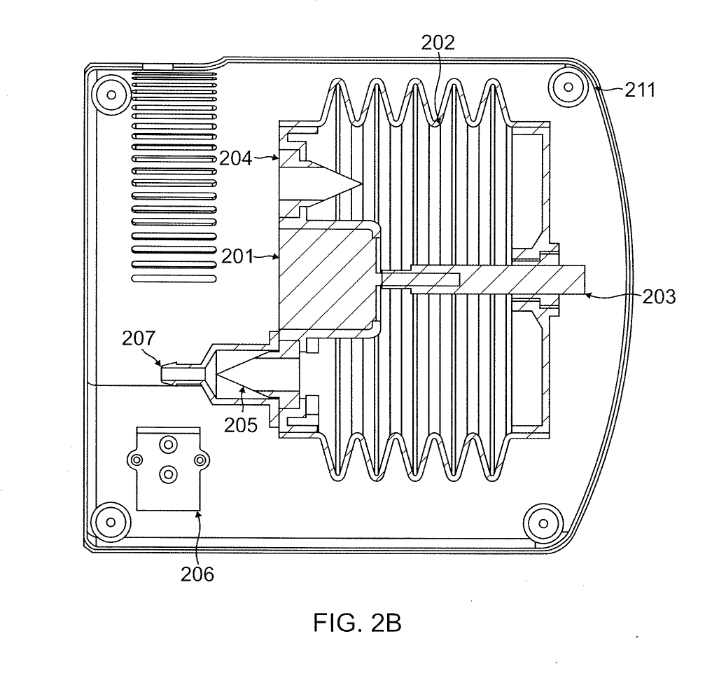

FIGS. 2A-2B provide an instance of a suitable gas generator of the system. FIG. 2A provides a side view of the gas generator wherein the gas generator is configured as a bellow. FIG. 2B provides a top-down view of the piston of FIG. 2A. As can be seen with respect to FIGS. 2A and 2B, a suitable gas generator 200 may be a bellows 202, which bellows 202 may be filled with the gas to be delivered. In such an instance, the volume of the bellows 202 defines an amount of the gas that can be delivered, which amount may be varied in accordance with the functioning of the bellows 202 in conjunction with the functioning of the motor 201 as controlled by the controller. For example, as depicted, the bellows 202 includes an gas inlet 204, such as a one way valve, a deformable cavity or reservoir within the bellows 202, and an outlet 207 to a patient interface, such as a nasal cannula. The gas to be delivered is drawn in through the gas inlet 208 thereby filling the deformable bellows 202. A casing 211 may also be provided for enclosing the elements of the gas generator may also be provided. The bellows 202 may be mounted to the casing 211 via a suitable bellows mounting fixture 209. A pressure transducer 206 or other sensor described as 106 may also be provided.

As can be seen with respect to FIG. 2B, the flow pulse generator may be operated by a motor, such as a servo-motor 201. The servo-motor may function by engaging a rotating a lead screw 203, which rotating screw 203 causes the bellows 202 to compress and expand. The motor 201 may be connected to a controller, such as a computer or a programmable logic controller. The controller controls the motor 201 which in turn controls the compression and/or expansion of the bellows 202 so as to deliver the desired flow pulse to the subject.

For instance, the controller may include programming containing a pulse control algorithm. The algorithm may take account of one or more inputs such as the lead screw pitch, the bellows geometry, and/or flow resistance through the system. In certain instances, when the subject inhales, a negative pressure may be detected, such as by the pressure transducer 206. When the negative pressure is detected by the pressure transducer 206 it triggers the servo motor 201 to turn the rotating screw 203 a given number of rotations, or parts thereof, thereby compressing the bellows to a given extent and delivering a pulse of gas. When the bellows compresses, gas travels through the outlet check valve 205 and towards the patient interface connection 207. When the bellows expand, gas enters the bellows chamber from the gas inlet 208 and through inlet check valve 204. The servo-motor 201 and lead screw 203 may be embedded within the bellows 202 to provide a small compact system to fit within the given enclosure 211. The bellows mount 209 may rigidly fix a lower end of the bellows 202 to the enclosure 211. Through holes in the mount for the check valves and servo-motor create an air tight seal and protect from contamination. Various tubing and an in-line filter within the tubing may be provided on the outlet to the patient interface. The pressure transducer port may connected via tubing to the nasal cannula at a barbed tee slightly downstream of the air outlet.

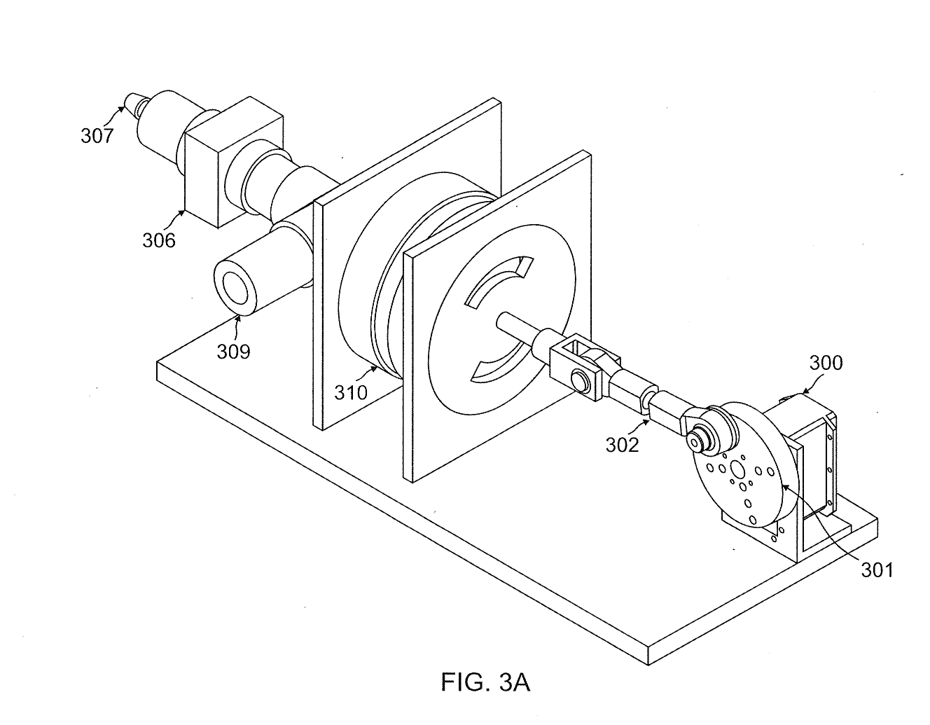

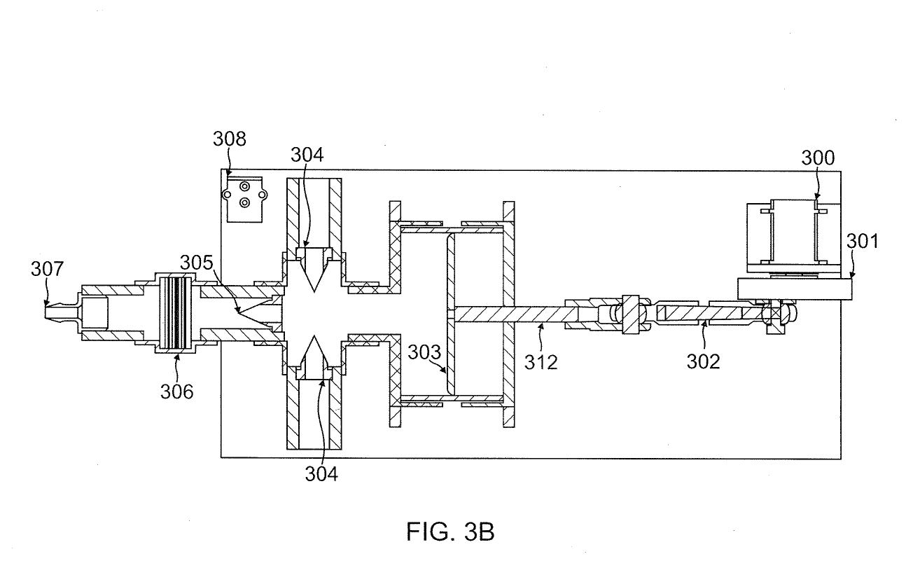

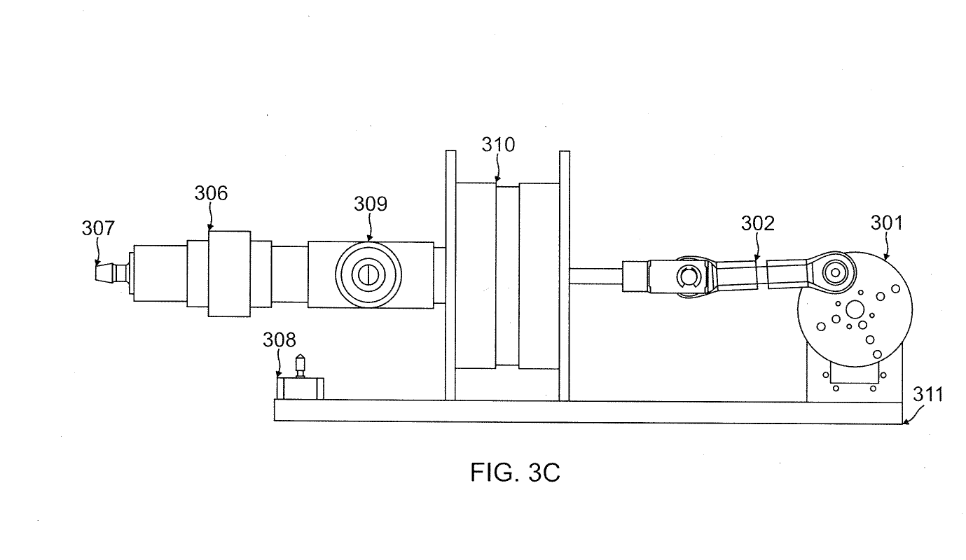

FIGS. 3A-3C provide another instance of a suitable gas generator of the system. FIG. 3A provides a perspective view of the gas generator wherein the gas generator is configured as a piston. FIG. 3B provides a top-down view of the piston of FIG. 3A. FIG. 3C provides a side-view of the piston of FIG. 3A. Accordingly, the gas generator of FIG. 3 is configured as a piston, which piston may be filled with the gas to be delivered. In such an instance, the volume of the piston chamber, e.g., reservoir, defines an amount of the gas that can be delivered, which amount may be varied in accordance with the functioning of the piston in conjunction with a motor as controlled by the controller.

For example, as depicted, the piston includes a gas inlet 304, such as a one way valve, a reservoir, an outlet 305, as well as piston arm 312. The gas to be delivered is drawn in through the gas inlet 304 thereby filling the reservoir. The reservoir may be defined by a length and a circumference the relationship between the two defining the volume of the delivery chamber. Given the interaction between the piston arm and the reservoir, the length of the reservoir is variable. Hence, as can be seen, the piston arm 312 articulates within the orifice of the reservoir so as to control the flow of the gas from reservoir. For instance, the motor 300 drives the positioning of the piston arm 304 relative to the reservoir 322.

As can be seen with respect to FIGS. 3A-3C, a piston configuration is provided wherein the piston includes a motor 300, such as a servo-motor, which motor 300 drives the piston 303. The drive wheel 301 and drive linkage 302 convert rotational motion of the motor 300 into linear motion of the piston 303. The motor 300 may be connected to a controller, such as a computer, or other programmable logic controller, which interacts with the piston to deliver the desired flow pulse to the subject. The controller may include a pulse control algorithm. The algorithm may take account of the drive wheel radius, linkage length, piston diameter, and flow resistance through the system and thereby determine the operation of the piston so as to deliver a specific predetermined amount of gas to the subject. When the subject inhales, a negative pressure may be detected by a pressure transducer 308, thereby triggering the motor 300 to deliver a pulse of gas. During a compression stroke, gas travels through the outlet check valve 305 and towards the subject interface connection 307. When the piston retracts, gas enters the piston chamber, e.g., reservoir, from the gas inlets 309 and through inlet check valves 304. The piston cylinder housing 310 clamps together to seal the compression side of the cylinder. The piston may use a spring-loaded PTFE seal. The pressure transducer port may be connected via tubing to a nasal cannula at a barbed tee slightly downstream of the air outlet 305. A filter 306 may be included between the air outlet 305 and the subject interface connection 307.

In manners such as these, the shape of the wave flow of the gas to be delivered to the subject may be modulated. The shape of the pulse may be modulated, for example, by modulating the bellow or piston or the like, such as by increasing or decreasing the acceleration, speed, deceleration, length, during which the bellows or piston move a given distance. For example, the ramp up speed of the delivery, size of the bolus, and duration of the delivery may all be modulated. The ramp up speed may be modulated by controlling how fast the bellows expands and compresses or how fast the plunger moves a given length of the reservoir. In certain instances, it may be desirable to have a rapid ramp up time, for instance, where it is desirable to activate the baroreceptors in the upper airways of a subject by delivering a sharp burst of gas to the subject at a given time point.

In such an instance, an initial rapid burst of gas can be delivered by the controller directing the motor's initial speed such that the bellows opens and closes rapidly, or the plunger travels a given initial distance in a short time period. The greater the opening and closing of the bellows or the greater distance traveled by the plunger over the shorter the period of time, the more rapid the ramp up speed will be and consequently the sharper the flow wave will be. In this manner, the baroreceptors of the upper airways may be activated by the delivery of a sharp burst of gas, which in turn will activate the underlying muscles of the airways, thus preventing the constriction of the airway and enhancing the patency thereof. It is to be noted that the sharpness of the delivery pulse should be sufficient to maintain patency of the airway without waking the subject up.

The size of the bolus may be modulated by directing the overall opening of the bellows or the distance the plunger travels relative to the length of the reservoir. The greater the opening or the further the plunger travels, the more gas is displaced thereby and the greater the size of the bolus. The size of the bolus to be delivered to the subject may provide a positive pressure to the airway and/or otherwise act as a pneumatic splint filling up the airway and preventing it from being constricted. The baroreceptors of the upper airways may also be activated by the delivery of a positive pressure of gas, which in turn will activate the underlying muscles of the airways thus preventing the constriction of the airway and enhancing the patency thereof. The size of the bolus should be such so as to pass the threshold for nerve stimulation, but not so great as to wake the subject up. The bolus may be of any suitable size, but in certain instances, will range from about 20 to about 250 ml, such as about 40 to about 200 ml, for instance, from about 60 ml to about 150 ml, including from about 72 or 75 ml to about 125 or about 100 ml.

In certain embodiments, the gas to be delivered may be present under higher pressure in a delivery reservoir, such as a gas tank. The reservoir may be operatively connected to an adjustable valve that is capable of being opened and closed in accordance with the commands of the controller. The amount of pressure as well as the opening and closing speeds as well as the length of the opening time of the active valve may all be controlled by the controller so as to shape the waveform of the gas flow. A traditional blower and active valve assembly may also be used. For instance, a pressure activated sensor may be provided in combination with an active valve, wherein the controller receives input from the pressure sensor and controls the opening and closing of the valve in response thereto. For example, a diaphragm may be provided in conjunction with the valve wherein the diaphragm is positioned so as to sense a change in pressure and communicate the same to the controller, which controller controls the opening characteristics of the valve and thereby controls the shape of the waveform. A bypass valve may also be included in embodiments wherein the blower is run continuously.

The duration of the pulse may be modulated by the overall time period the bellows open and close or the plunger moves relative to a given length of the reservoir. The longer the bellows operate or the longer it takes the plunger to move a given length of the reservoir the greater the duration of delivery will be. The duration of the time period may also enhance the patency of the upper airway by reducing the opportunity for constriction occurring during a given breathing cycle.

In certain embodiments, the timing of the delivery may also be controlled such that the gas may be delivered continuously, periodically in response to a sensed need, and/or cyclically in accordance with a predetermined pattern. The triggering event may also be adjusted so as to deliver the gas in accordance with the subject's need and/or in accordance with a predetermined pattern. For instance, the sensitivity of the pressure sensor may be adjusted and/or the timing of the trigger may be adjusted. For example, the trigger may be adjusted so as to ensure the delivery of a pulse of gas within 500 ms of the initiation of inspiration, so as to ensure maximum saturation during the breathing cycle. This delay may be adjusted as necessary to meet the needs of the subject at any given time period. In certain embodiments, the device may further be configured to auto-trigger the delivery of a pulse of gas if a subject stops breathing, e.g., an apneic event is sensed, such as by the lack of a trigger within a given predetermined time period. Because subjects will differ with respect to the anatomy of their airways and/or the severity of their condition, one or more of the above may be adjusted so as to ensure the gas is delivered in a manner that meets the needs of each individual subject.

As set forth above, an aspect of the disclosure is the delivery of a gas to a subject wherein the gas is delivered intermittently in accordance with a sensed need, such as by sensing the onset of a breathing event. In another aspect of the disclosure a gas may be delivered to a subject in accordance with a predetermined model, such as a model developed as the result of a sleep study, e.g., a titration study. In certain implementations, the gas delivery device may be configured for switching from an intermittent delivery model to a predetermined delivery or continuous delivery model, for instance, in response to a predetermined event. In certain instances, the system may be configured such that if an apnea event occurs a default backup delivery rate is initiated. A backup delivery rate may be a prescribed continuous delivery rate. A suitable backup delivery rate may be, for instance, at about 15 breaths per minute. Once normal breathing has been resumed the system may then return to breath activation mode.

For instance, in one implementation, the device may be configured for delivering the gas intermittently in response to the subject's sensed breathing needs. However, if an apneic event occurs, the device may be configured for switching from intermittent mode to a predetermined delivery or continuous delivery mode. For example, in accordance with the methods disclosed herein, a gas delivery system may be employed at a clinical treatment facility or home, etc., so as to determine a model for gas delivery, which model may be employed in a non-clinical setting, such as at home. As is known in the art, such a model is typically determined as a result of a sleep study that is performed in a clinical setting, although with various modifications disclosed herein, such a study may be performed at home, for instance, by the user, in accordance with parameters set forth by suitable auto-titration programming of the device.

Accordingly, a sleep study employing a device of the disclosure may be carried out so as to develop one or more models for gas delivery. For instance, a sleep study may be performed to determine a model for the optimal waveform characteristics of the gas to be delivered to a particular subject, e.g., while sleeping, so as to prevent and/or treat an unhealthy condition, such as sleep apnea. In this manner, predicted ideal waveform characteristics, with respect to trigger time, the rise, sharpness, volume/pressure, and duration of gas delivery, and the like, may be determined for the individual. Once determined or otherwise predicted, these characteristics may be employed to shape the waveform to be delivered by the device. For example, the dimensions as to onset (e.g., triggering), rise, rise time, volume, pressure, duration, and the like, may be made accessible to the controller, which controller may then use these dimensions to shape the wave of the gas to be delivered to the subject, e.g., by controlling the functioning of one or more of a motor, gas generator, valve, and the like.

As set forth above, once determined, the gas may be delivered in accordance with these dimensions intermittently, for instance, in response to the onset of a breathing event. However, in certain instances, the predicted ideal may not In fact be the actual ideal and an apneic event may still occur. For instance, the pressure or other characteristic of the gas flow may not be sufficient to maintain the patency of the airways of the subject and consequently one or more of the waveform characteristics may need to be modified so as to ensure such patency. In such an instance where an apneic event is sensed or otherwise determined, the device may be configured for one or both of switching to a continuous mode and for making corrections to one or more of the waveform characteristics currently being employed in delivering the gas to the individual. For instance, once an apneic event is determined the device may switch to a correction mode wherein a sharper rise, a higher volume, a higher pressure, and/or a longer duration of gas is delivered than what was previously being delivered so as to prevent any further apneic events from occurring. Of course, any of these characteristics may be decreased as well if it is sensed that the subject is waking up because the settings are too high.

In certain embodiments, the gas with the adjusted characteristics may be delivered, for instance, in response to a sensed need of the subject. In other embodiments, however, it may be delivered continuously or periodically in accordance with a predetermined timing until a more regular breathing pattern is determined. For instance, if an apneic event is determined, e.g., if no breath is sensed within a given time period, then an automatic bolus may be delivered or the mode of delivery may be switched. For example, an increasing series of boluses may be delivered, such as in an auto correction mode, until the apneic events cease, or the delivery mode may be switched such that the gas may be delivered continuously, for instance, until adjusted by the user, e.g., upon awaking, or may be delivered continuously until a more normalized breathing pattern is determined.

Alternatively, the gas delivery mode may be switched such that the gas may also be delivered periodically, such as in accordance with a predetermined timing of delivery, until a more normalized breathing pattern is determined. For instance, the subject's breathing pattern while sleeping may be determined during a sleep study and the average timing of inhalation and exhalation may be determined, thus the gas may be delivered periodically at a time period that is estimated to coincide with the inhalation phase determined during the sleep study. Thus, the system may be configured for delivering a gas during an inhalation portion (beginning) of breathing, and not during exhalation, e.g., when a user is breathing out.

In one embodiment, the gas may also be delivered in accordance with a running determination pattern. For instance, the controller may be associated with one or more sensors wherein together they function to receive the respiratory signals of the subject in situ in a manner sufficient to determine and distinguish the running or ongoing inhalation pattern and the running or ongoing exhalation pattern. An average period for inhalation and exhalation may be determined from this data, e.g., over several respiratory cycles, and thereby determine when during the average inhalation phase the gas should be delivered and/or how to shape the waveform of the gas to be delivered.

An apneic event may be determined in any manner known in the art. For instance, using routine procedures during the sleep study, an apneic event may be defined for the subject, e.g., as a cessation of breathing for a number of times in a given time period, or a cessation of breathing for a prolonged time period, or the like. Consequently, the defined apneic event may be employed by the device such that if the defined event is determined to have occurred during the subject's use of the device, then the device may automatically switch into a continuous and/or correction mode, e.g., a predetermined or running determination delivery mode, so as to self-correct. In certain instances, such self-correction may result in the device changing one or more characteristics of the waveform of the gas being delivered so as to prevent any further apneic events. This may be performed automatically in a manner such that the corrections are made without causing the subject to wake up. For instance, changes may be made incrementally to any of the various wave form characteristics, the effects of the changes may be determined, e.g., via suitable sensors, and may be repeated as necessary until no further apneic events are determined or otherwise sensed.

For example, in one instance, an initial delivery pattern of gas to a subject may not have a sharp enough rise time, high enough pressure, and/or long enough duration so as to prevent an apneic event from occurring. Such an event may be determined to occur in situations where there is only a partial block of the airways, and thus, a larger pressure or sharper rise is beneficial for ensuring that an apneic event does not occur. The device may therefore be configured to sense or predict the apneic event and to make corrections to the gas being delivered in such a manner that the corrections are made automatically, for instance, before the subject is woken up due to apnea. Of course, if the waveform characteristics are too high, the subject may adjust the level of the characteristics manually. In certain embodiments, it may be determined that an oscillation event may be beneficial, in which instance, the delivery of the gas wave may be configured such that at the peak of delivery the waveform oscillates, for instance, in a manner so as to activate one or more baroreceptors of the airway. This may be accomplished, for instance, by configuring the bellows or piston to pulsate at a rapid pace during the top end of the delivery phase or in other like manner. Such adjustments and modulations to the pulse of gas being delivered may be useful, for instance, in situations where a subject's tidal volume changes, e.g., during the course of sleeping and/or in response to the subject's emotional state.

Accordingly, in one instance, the methods of the disclosure include determining a model for delivering a gas wave in a series of pulses and/or may include correcting the same as necessary without waking the subject from sleep. Such a titration method may be performed automatically by the device, in accordance with the programming thereof, or in accordance with a predetermined pattern, or may be performed in conjunction with a sleep study, which patterns once determined may be made available to the programming of the device and be employed thereby to make automatic corrections in the manner described herein.

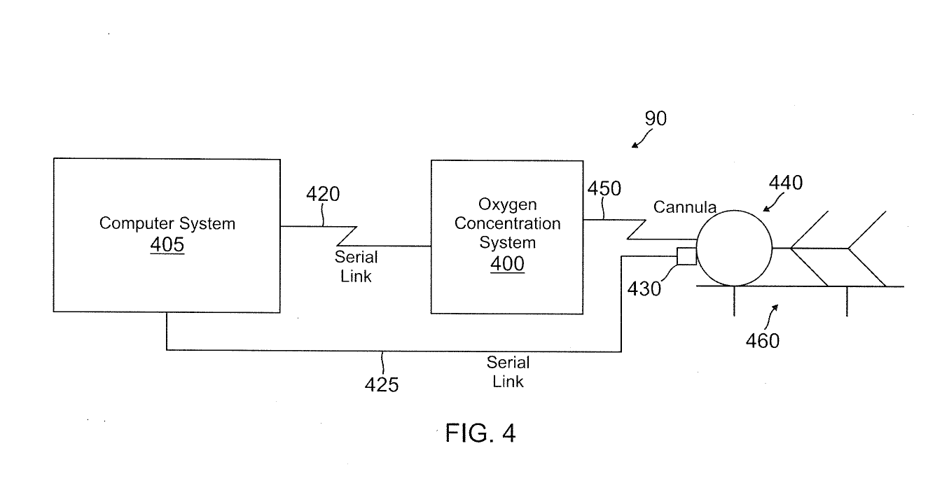

FIG. 4 provides an exemplary titration system 490 that may be employed in performing a sleep study for a subject 440 believed to be in need thereof. As disclosed herein, the sleep study may be performed so as to determine one or more predicted ideal wave form characteristics for the delivery of a gas to a subject, such as for the purpose of preventing and/or treating an unhealthy condition, e.g., sleep apnea, for instance, so as to maintain the patency of the subject's airways. The titration system 490 may include a gas generating system 400 that is operably linked via a suitable linkage 420 to a computer system 405. The gas generating system 400 may be a pulse and/or continuous mode gas generating system and may include one or more of a gas generator, motor, energy source, control unit, and sensor, as described above. The gas generating system 400 may be associated with cannula 450, which cannula is specially configured for interfacing with the subject 440 for the delivery of the gas from the gas generating system 400 to the subject 450. The gas generating system 400 delivers the gas, such as in a pulsed or continuous mode, to the subject 440 while the subject is sleeping.

Sensor(s) 430 may further be included, wherein the sensor(s) 430 is operably linked to the computer system 405, via a suitable linkage 425, so as to provide feedback to the computer system pertaining to the subject's breathing and state of sleep. This or any other linkage may be via a cable or wireless connection. The sensor(s) 430 may be configured for measuring the subject's breathing so as to determine whether the subject stops breathing and for how long. The sensors may also be configured to monitor sleep state and be of a type commonly used on polysomnography.

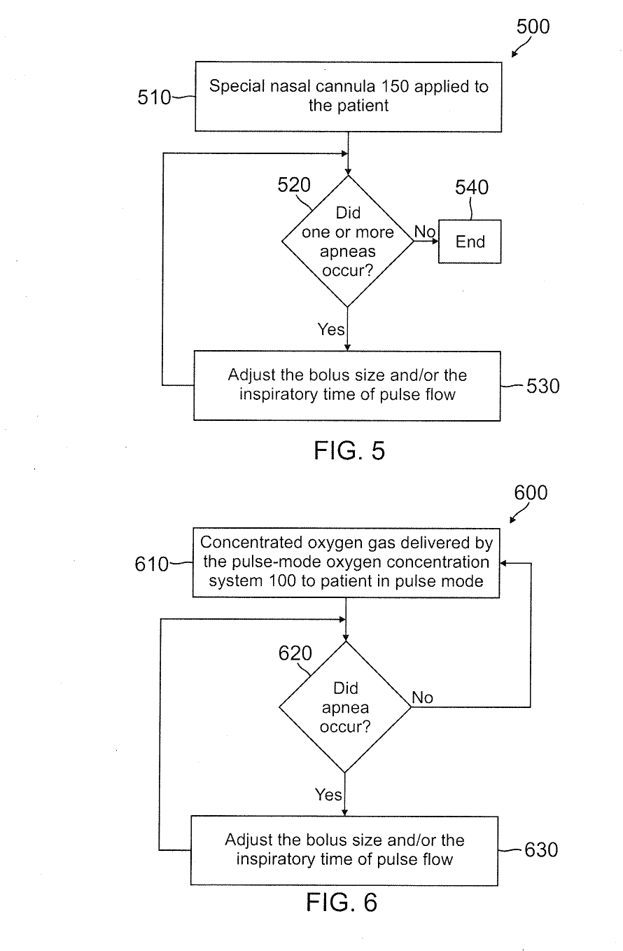

As set forth above, the pulsed air/concentrated oxygen system 100, may be configured for adjusting inspiratory time as well as the characteristics of a delivered bolus of gas. Accordingly, in FIG. 5 the titrating system 490 is employed in performing a titration method during a sleep study 500 so as to determine one or more models for the delivery of a gas to the subject. For instance, a sleep study may be performed to determine one or more ideal characteristics of a waveform of gas to be delivered to a subject. Once titrated, the pulsed air/concentrated oxygen system 100 may be employed for the treatment and/or prophylaxis of sleep apnea, such as mild-to-moderate sleep apnea. The method 500 may be performed in a sleep clinic where the subject 440 is lying on a bed 460 and is being monitored by one or more clinicians (hereinafter “clinician”).

As set forth above, the programming of the controller may include an auto-titration mode and thus the device may be configured so as to allow a subject to titrate the device while at home, or elsewhere outside of a sleep clinic, by following the instructions provided by the programming of the controller. Hence, the device may be configured for determining when an apneic event has occurred, recording the same, and responding thereto by changing delivery time and/or one or more of the characteristics of the waveform of the gas being delivered, e.g., automatically. The following steps, therefore, may be performed by a clinician, someone else following the instructions provided by the device itself, or by a subject in conjunction with the programming of the device.

Accordingly, at step 510, a special nasal cannula 450 such as that described herein below is applied to the subject. At step 520, a determination is made as to whether one or more apnea events occur. If it is determined that one or more apnea events occur, e.g., by the clinician or other, then at step 530, a characteristic of the waveform of the gas being delivered, e.g., bolus shape, size and/or the inspiratory time of the pulse flow, is adjusted, and the process returns to step 520. If it is determined that little or no apnea events, then the method 500 ends at 540. These steps may be repeated until it is determined that no further significant apnea events occur. Thus, in the titration method 500, the clinician or other, monitors the subject's 440 breathing during sleep for apnea events and the device is titrated to the point where little or no recurrent apnea events are determined.

A backup pulse flow rate may also be determined and/or prescribed for the determined bolus shape, size, and/or the inspiratory time. The backup pulse flow rate is an automatic pulse flow rate delivered to the subject 440 by the pulsed air/concentrated oxygen system 100 when the pulsed air/concentrated oxygen system 100 determines a subject has an apnea event while sleeping (e.g., at home) or is otherwise in need thereof.

For instance, during a titration, the subject's breathing may be tracked so as to determine a baseline breathing pattern. The baseline breathing pattern may include both a pattern characterizing the inhalation phase and exhalation phase of the subject's breathing cycle. In one instance, the device may be configured such that during use, the subject's current actual breathing pattern is determined and is compared to the determined baseline breathing pattern. If the subject's current actual breathing pattern matches the subject's baseline breathing pattern, the pulse of gas is delivered periodically at the inhalation stage of the subject's breathing pattern. However, if a break in the subject's breathing pattern is determined, for example, wherein the subject's current actual breathing pattern does not match the subject's baseline breathing pattern, such as during an apnea event, the gas is delivered in accordance with a backup pulse flow rate.

As set forth above, the characteristics of the waveform of the gas flow may be adjusted, e.g., automatically, so as to reduce the occurrence of apenic events. Hence, when a cessation of breathing is detected e.g., the subject stops breathing, the gas may be delivered in a series of pulses such as in accordance to a preset pattern, which pattern is designed to increase one or more of the characteristics of the gas flow waveform, e.g., rise time, amplitude, size, inspiratory time length, and the like, so as to diminish the occurrence of apneic events. Once the occurrence of apneic events has been reduced, the gas may then be delivered in accordance with this new pattern. For instance, the pulses may be delivered continuously in accordance with a preset pattern until the subject's current actual breathing pattern matches the subject's baseline breathing pattern. A suitable backup pulse flow rate, such as that prescribed by a physician, may be from about 10 to about 20 bpm.

Accordingly, in one aspect, a method for treating an unhealthy sleep condition is provided wherein the method includes examining the subject's sleeping pattern, for instance, so as to determine the cycle as to when the subject inhales and when the subject exhales, and/or the subject's tidal volume while sleeping, as well as determining the occurrence of one or more sleep apneic events. The examination may include delivering a quantity of gas to the subject while sleeping and determining the characteristics of the waveform of the gas being delivered that appear to be beneficial to the subject, e.g., that reduce the number, size, and/or frequency of apnea events.

Therefore, the gas to be delivered may be delivered at the onset of a determined inhalation event that is sensed or predetermined as a result of the sleep study. For example, the gas may be delivered in accordance with the subject's determined sleeping pattern, for instance, at the onset of a sensed or expected inhalation phase, which inhalation phase may be determined by a sleep study, so as to treat an unhealthy symptom or condition. A condition or symptom associated therewith may be one or more of obstructive sleep, sleep apnea, snoring, asthma, allergies, inflammation, hypertension, cardiovascular complications, stroke, type II diabetes, fatigue, sleepiness, and the like. Additionally, the gas may be delivered for the purpose of delivering an aroma, e.g., for aroma therapy, and/or for delivering a nebulized medicament, e.g., a pharmaceutical. In certain embodiments, the gas may be delivered for the purpose of synchronizing the breathing of the subject, stimulating the hypoglossal nerve, stimulating a subject's baroreceptors, and/or for delivering a positive pressure to the pharynx of the subject.

With reference to FIG. 6, a method 600 of using the pulsed air/concentrated oxygen system 100 for treating an unhealthy condition, such as mild-to-moderate sleep apnea, will be described. First, at step 610, air/concentrated oxygen gas is delivered by the pulsed air/concentrated oxygen system 100, such as in pulse mode. The pulsed air/concentrated oxygen system 100 senses an inspiratory trigger and delivers a bolus of gas in response thereto. The delivery of gas may be breath activated or pressure activated, or the like. For instance, using one or more of a breath sensor, a breath inspiration sensor, and/or pressure sensor, the device senses each time the subject takes a breath and then delivers a pulse of gas. In certain instances, the gas is pressure activated and the delivery of the gas is contingent upon detecting a drop in pressure. In other instances, a rise in pressure inhibits the delivery of the gas.

At step 620, a determination is made as to whether an apnea event occurred. If an apnea event has not occurred, control goes back to step 610. If an apnea event has occurred (e.g., a subject stops breathing), control goes to step 630, and the pulsed air/concentrated oxygen system 100 automatically defaults to the prescribed back-up rate (e.g., as determined above) at the prescribed bolus size and/or inspiratory time settings. The pulsed air/concentrated oxygen system 100 may deliver air or concentrated oxygen to the subject 440 at the back-up rate until it is determined that the subject has resumed spontaneous breathing. Once the subject 440 resumes spontaneous breathing, the subject's breath rate will trigger the pulsed air/concentrated oxygen system 100 and drive the respiratory rate.

In certain instances, the device may be configured for monitoring a subject's use of the device, for instance, in accordance with a prescribed use. The device may record usage data, such as data that may be provided to an insurance carrier so as to substantiate use parameters. Such use data may be communicated to a user or third party, such as a physician, insurance carrier, or the like, in any suitable manner, such as via wireless or wire communication. Accordingly, the device may be configured for wifi, wwan, Bluetooth, or other suitable wireless communication. The system may be configured for communicating this and/or other information periodically or continuously, such as in accordance with a set schedule, for instance, every 30, 60, 90 days or the like. In certain instances, by monitoring usage of the device compliance can be shown. For example, by tracking pulse deliveries, it can be determined whether and to what extent the subject was triggering pulses and therefore using the device and thereby determining compliance. For instance, compliance can be determined by a doctor and/or the like and may be about 4 or 5 hours a night and 5 or more nights a week.

In one aspect a high flow cannula system is provided, including a cannula for use with a gas delivery device and/or system of the disclosure. The cannula system is configured such that it provides a low flow resistance. In certain instances, a low flow resistance may mean that the resistance may have a pressure drop that is less than about 1.2 psid at 40 liters per minute (lpm). For instance, the resistances can be between about 0.1 and about 4 psid, such as about 0.5 and about 3 psid, including about 1 and about 2 psid when flowing 40 lpm air. The bigger the inside diameter the lower the pressure drop. The cannula system may include a user interface on one end, a device interface positioned at an opposing end, and transfer tubing there between. In certain instances, the user interface is configured as an open system. However, in certain instances, it may be configured as a closed system and include a typical mask configuration.

Where the cannula system is configured as an open system, the user interface may include a plurality of nostril prongs that are configured for interacting with a plurality of nubbins that are specifically designed to be positioned within or adjacent to the nostrils of the user. The nubbins may come in different shapes and sizes and may be configured for being removably attached to the nostril prongs of the user interface. For instance, the nubbins may be sized and shaped such that by being attached to the nostril prongs they may change the resistance at the user interface of the cannula system. In certain instances, the nubbin can have different size openings. For example, bigger openings have less of a pressure drop than smaller openings. Therefore, the desired pressure drop may be determined and implemented by configuring the openings of the nubbins accordingly.

For example, the nubbin pairs may have a mushroom shape designed to fit comfortably within the nostrils of a user and may have an opening of a particular diameter that allows the passage of the gas from the device to the user. The size of the mushroom head and the size of the opening may be varied in such a manner to increase or decrease the resistance at the user interface and thereby modulate the resistance of the cannula system. For instance, the size of the mushroom head may be increased and the size of the opening may be decreased so as to increase resistance in the system. Likewise the size of the mushroom head may be decreased and the size of the opening may be increased so as to decrease resistance in the system.

In certain implementations, several sets of nubbins may be provided wherein each nubbin pair provides the user interface with a different resistance, such as a low, medium, and high resistance. In such an instance, the nubbins may be color coded so as to distinguish the differences between them with respect to the different sizes and/or resistances. Additionally, the nubbins may have a variety of different shapes and sizes so as to fit comfortably in a variety of different nostrils, such as small, medium, and large sizes. In certain instances, the nubbins may be configured to completely occlude the nostril passage when appropriately positioned therein. In other instances, the nubbins may be configured so as to not completely occlude the nostril passage when positioned therein. The nubbins may be configured for a single use and/or may be disposable or recyclable. Although the nubbins may be configured for being associated with the nostril prongs, in certain instances they may be configured for being associated elsewhere on the system, such as along the length of the transfer tubing. Further, in certain instances, the nubbins are configured to be disposable, for instance, after a single use. The device interface may be configured for interacting with an outlet portion of the gas delivery system so as to join the cannula system to the device.

The material used can also affect pressure drop. For example, some materials are “slippery” and others “rough.” The transfer tubing may be fabricated out of any suitable material and may have any suitable diameter. However, in certain instances, the material of the tubing and the diameter may be configured for decreasing or increasing resistance. In certain instances, silicone tubing may be used so as to provide a higher relative pressure drop, in other instances, polyurethane tubing may be used to provide a lower relative pressure drop. In certain instances, it is desirable to decrease pressure drop in general to allow the fastest delivery of a pulse.

In one aspect, a kit may be provided. The kit may include one or more of a gas generating and/or concentrating device, e.g. an oxygen concentration system, a computer system, sensor, and/or nasal cannula system including one or a plurality of nubbins, as described above. In certain instances, one or more of the kit items may be packaged together, for instance, in a single packaged system. A set of instructions setting forth how to configure and use the system as well as its components may also be provided as a component of the kit.

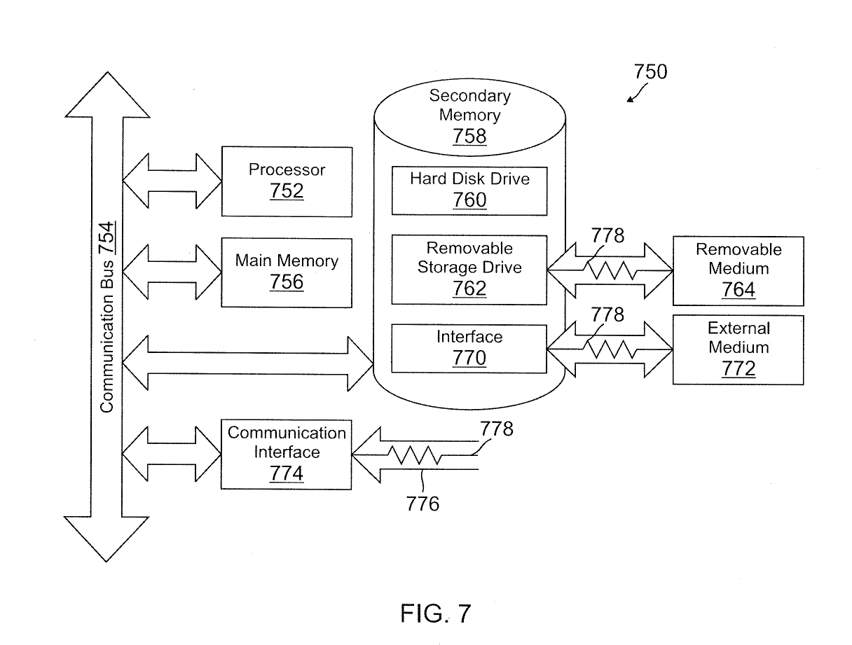

FIG. 7 is a block diagram illustrating an example computer system 750 that may be used in connection with the embodiment of the computer system 405 and/or control unit 110 described herein. However, other computer systems and/or architectures may be used, as will be clear to those skilled in the art.

The computer system 750 preferably includes one or more processors, such as processor 752. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 752.

The processor 752 is preferably connected to a communication bus 554. The communication bus 754 may include a data channel for facilitating information transfer between storage and other peripheral components of the computer system 550. The communication bus 754 further may provide a set of signals used for communication with the processor 752, including a data bus, address bus, and control bus (not shown). The communication bus 754 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

Computer system 750 preferably includes a main memory 756 and may also include a secondary memory 758. The main memory 756 provides storage of instructions and data for programs executing on the processor 752. The main memory 556 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 758 may optionally include a hard disk drive 560 and/or a removable storage drive 762, for example a floppy disk drive, a magnetic tape drive, a compact disc (“CD”) drive, a digital versatile disc (“DVD”) drive, etc. The removable storage drive 762 reads from and/or writes to a removable storage medium 764 in a well-known manner. Removable storage medium 764 may be, for example, a floppy disk, magnetic tape, CD, DVD, etc.

The removable storage medium 764 is preferably a computer readable medium having stored thereon computer executable code (i.e., software) and/or data. The computer software or data stored on the removable storage medium 764 is read into the computer system 750 as electrical communication signals 778.

In alternative embodiments, secondary memory 758 may include other similar means for allowing computer programs or other data or instructions to be loaded into the computer system 750. Such means may include, for example, an external storage medium 772 and an interface 770. Examples of external storage medium 772 may include an external hard disk drive or an external optical drive, or and external magneto-optical drive.

Other examples of secondary memory 758 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage units 772 and interfaces 770, which allow software and data to be transferred from the removable storage unit 772 to the computer system 750.

Computer system 750 may also include a communication interface 574. The communication interface 774 allows software and data to be transferred between computer system 750 and external devices (e.g. printers), networks, or information sources. For example, computer software or executable code may be transferred to computer system 750 from a network server via communication interface 774. Examples of communication interface 774 include a modem, a network interface card (“NIC”), a communications port, a PCMCIA slot and card, an infrared interface, and an IEEE 1394 fire-wire, just to name a few.

Communication interface 774 preferably implements industry promulgated protocol standards, such as Ethernet IEEE 802 standards, Fiber Channel, digital subscriber line (“DSO, asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 774 are generally in the form of electrical communication signals 778. These signals 778 are preferably provided to communication interface 774 via a communication channel 776. Communication channel 776 carries signals 778 and can be implemented using a variety of wired or wireless communication means including wire or cable, fiber optics, conventional phone line, cellular phone link, wireless data communication link, radio frequency (RF) link, or infrared link, just to name a few.

Computer executable code (i.e., computer programs or software) is stored in the main memory 756 and/or the secondary memory 758. Computer programs can also be received via communication interface 774 and stored in the main memory 756 and/or the secondary memory 758. Such computer programs, when executed, enable the computer system 750 to perform the various functions of the present invention as previously described.

In this description, the term “computer readable medium” is used to refer to any media used to provide computer executable code (e.g., software and computer programs) to the computer system 750. Examples of these media include main memory 756, secondary memory 758 (including hard disk drive 760, removable storage medium 764, and external storage medium 772), and any peripheral device communicatively coupled with communication interface 774 (including a network information server or other network device). These computer readable mediums are means for providing executable code, programming instructions, and software to the computer system 750.

In an embodiment that is implemented using software, the software may be stored on a computer readable medium and loaded into computer system 750 by way of removable storage drive 762, interface 770, or communication interface 774. In such an embodiment, the software is loaded into the computer system 750 in the form of electrical communication signals 778. The software, when executed by the processor 752, preferably causes the processor 752 to perform the inventive features and functions previously described herein.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in any following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

The systems and methods disclosed herein may be embodied in various forms including, for example, a data processor, such as a computer that also includes a database, digital electronic circuitry, firmware, software, or in combinations of them. Moreover, the above-noted features and other aspects and principles of the present disclosed embodiments may be implemented in various environments. Such environments and related applications may be specially constructed for performing the various processes and operations according to the disclosed embodiments or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques.

The systems and methods disclosed herein may be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Although the description above refers to a client and a server, other frameworks and architectures may be used as well. For example, the subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

As used herein, the term “user” may refer to any entity including a person or a computer.

The foregoing description is intended to illustrate but not to limit the scope of the invention, which is defined by the scope of the appended claims. Other embodiments are within the scope of the following claims.

What is claimed:

1. A method for providing a gas to a subject in need thereof, the method comprising sensing onset of the subject's inhalation; and delivering a pulse of gas in response to the subject's inhalation.

2. The method of claim 1, wherein the method further comprises treating the subject for a symptom associated with one or more of obstructive sleep, sleep apnea, snoring, asthma, allergies, inflammation, hypertension, cardiovascular complications, stroke, type II diabetes, fatigue, and sleepiness.

3. The method of claim 1, wherein the method comprises providing aroma therapy to the subject.

4. The method of claim 1, wherein the method is practiced for the purpose of delivering a positive pressure to the pharynx of the subject.

5. The method of claim 1, wherein the method is practiced for the purpose of stimulating the hypoglossal nerve.

6. The method of claim 1, wherein the method is practiced for the purpose of stimulating a subject's baroreceptors in a subject.

7. The method of claim 1, wherein the delivering of the gas is pressure activated and the delivery of the gas is contingent upon detecting a drop in pressure.

8. The method of claim 7, wherein the method further comprises sensing onset of the subject's exhalation.

9. The method of claim 8, wherein the method further comprises ceasing the delivery of gas during exhalation.

10. The method according to claim 9, wherein a rise in pressure inhibits the delivery of the gas.

11. The method of claim 1, wherein the method further comprises tracking the subject's breathing so as to determine a baseline breathing pattern.

12. The method of claim 11, wherein the baseline breathing pattern comprises both a pattern characterizing the inhalation phase and exhalation phase of the subject's breathing cycle.

13. The method of claim 11, further comprising detecting a current actual breathing pattern and comparing the current actual breathing pattern to the baseline breathing pattern.

14. The method of claim 13, wherein when the subject's current actual breathing pattern matches the subject's baseline breathing pattern, the pulse of gas is delivered periodically at the inhalation stage of the subject's breathing pattern.

15. The method of claim 14, wherein the method further comprises detecting a break in the subject's breathing pattern such that the subject's current actual breathing pattern does not match the subject's baseline breathing pattern.

16. The method of claim 15, wherein the break in the subject's normal breathing pattern comprises a cessation in breathing.

17. The method of claim 16, wherein when a cessation of breathing is detected the gas is delivered in a series of pulses according to a preset pattern.

18. The method of claim 17, wherein the series of pulses are delivered continuously in accordance with the preset pattern until the subject's current actual breathing pattern matches the subject's baseline breathing pattern.

19. The method of claim 18, wherein once the subject's current actual breathing pattern matches the subject's baseline breathing pattern, the pulse of gas is delivered periodically at the inhalation stage of the subject's breathing pattern.

20. A method for treating an unhealthy sleep condition in a subject expected to be suffering there from, comprising:
examining the subject's sleeping pattern so as to determine the cycle as to when the subject inhales and when the subject exhales while sleeping;
providing the subject with a device configured for both delivering a quantity of gas to the subject in response to the onset of an inhalation event and for delivering the gas in accordance with the determined sleeping pattern; and
instructing the subject to use the device while sleeping.

21. The method of claim 20, wherein the sleep condition comprises one or more of obstructive sleep, sleep apnea, snoring, asthma, allergies, and inflammation.

22. A device for delivering a quantity of gas to a subject in direct response to the subject's need, the device comprising:
a gas generator, for generating a quantity of gas and for delivering the same to the subject;
a sensor for sensing the subjects need for the gas, and
a controller for controlling the delivery of the gas to the subject in response to the subject's sensed need for the gas.

23. A low flow resistance cannula system for coupling to a gas delivery device, comprising:
a user interface having a set of nostril prongs;
transfer tubing; and
a gas delivery device interface configured for coupling the user interface to the gas delivery device, wherein the low flow resistance cannula system comprises a pressure drop that is between about 0.1 and about 4 psid when the gas delivery device is delivering a gas that is flowing through the cannula system at 40 liters per minute (lpm).

24. The low flow resistance cannula system of claim 23, wherein the pressure drop is between about 0.5 and about 2 psid when a delivery gas is flowing there through at 40 liters per minute (lpm).

25. The low flow resistance cannula system of claim 23, wherein the resistances is less than about 1.2 psid at 40 lpm.

26. The low flow resistance cannula system of claim 23, further comprising a plurality of nubbins connectedly associated with the nostril prongs.

27. The low flow resistance cannula system of claim 26, wherein the nubbins are configured for being removably attached to the nostril prongs of the user interface.

28. The low flow resistance cannula system of claim 27, wherein a plurality of sets of nubbins are provided.

29. The low flow resistance cannula system of claim 28, wherein each of the plurality of sets nubbins has a different shape and size.

30. The low flow resistance cannula system of claim 29, wherein the sets of nubbins are provided in a small, medium, and large size.

31. The low flow resistance cannula system of claim 30, wherein the plurality of sets of nubbins are color coded dependent on the size.

32. The low flow resistance cannula system of claim 28, wherein each of the plurality of sets of nubbins has a different resistance.

33. The low flow resistance cannula system of claim 32, wherein the nubbins are provided in a low, medium, and high resistance.

34. The low flow resistance cannula system of claim 33, wherein the plurality of sets of nubbins are color coded dependent on the resistance.

35. The low flow resistance cannula system of claim 26, wherein each nubbin comprises a mushroom head.

36. The low flow resistance cannula system of claim 35, wherein each mushroom head shaped to fit comfortably within the nostrils of a user.

37. The low flow resistance cannula system of claim 35, wherein the size of the mushroom head is increased so as to increase resistance in the system.

38. The low flow resistance cannula system of claim 35, wherein the size of the mushroom head is decreased so as to decrease resistance in the system.

39. The low flow resistance cannula system of claim 35, wherein the nubbins are configured for single use.

40. The low flow resistance cannula system of claim 39, wherein the nubbins are recyclable or disposable.

41. The low flow resistance cannula system of claim 23, wherein the transfer tubing has a diameter configured for decreasing resistance so as to provide a higher relative pressure drop.

42. The low flow resistance cannula system of claim 23, wherein the transfer tubing comprises silicone.

43. The low flow resistance cannula system of claim 23, wherein the transfer tubing comprises polyurethane.
